(19) United States
(12) Patent Application Publication
(10) Pub. No.: US 2023/0027503 A1
(43) Pub. Date: Jan. 26, 2023

(54) SYSTEM AND METHOD OF BIOCHEMICAL MOLECULE SYNTHESIS AND DETECTION IN A POINT OF COLLECTION SETTING

(71) Applicant: Genalyze LLC, Middletown, DE (US)

(72) Inventors: Chen-Yu Liu, Middletown, DE (US); Hui-Ju Tsai, Middletown, DE (US)

(73) Assignee: Genalyze LLC, Middletown, DE (US)

(21) Appl. No.: 17/869,367

(22) Filed: Jul. 20, 2022

Related U.S. Application Data
(63) Continuation-in-part of application No. 17/395,604, filed on Aug. 6, 2021.
(60) Provisional application No. 63/223,966, filed on Jul. 20, 2021.
(60) Provisional application No. 63/223,972, filed on Jul. 21, 2021.
(60) Provisional application No. 63/274,439, filed on Nov. 1, 2021.
(60) Provisional application No. 63/328,058, filed on Apr. 6, 2022.

(51) Int. Cl. B01L 7/00

(52) U.S. Cl. CPC B01L 7/52; B01L 2400/0487

(57) ABSTRACT

A system for nucleic acid amplification is to synthesize amplified target nucleic acids or determine the presence of target nucleic acid. The mobile device of the system implements with an interface for controlling the reaction as well as optionally recording or delivering the reaction results or protocols to a cloud for sharing. In addition, current invention also discloses an airborne molecule detector integrating both air sampler and biochemical analysis component. The device can monitor the bioaerosols on real time. The reaction product can be used for nucleic acid sequencing as well. Furthermore, a pH test strip is used to replace a halochromic agent in a reaction mix for determining the nucleic acid amplification.

### CROSS-REFERENCE TO RELATED APPLICATIONS

This application claims priority of U.S. Provisional Application 63/223,972 entitled “System and method of nucleic acid amplification for point of collection”, filed Jul. 21, 2021, U.S. Provisional Application 63/223,966 entitled “System and method of nucleic acid amplification for point of collection”, filed Jul. 20, 2021, U.S. Non provisional application Ser. No. 17/395,604 entitled “System and method of nucleic acid amplification for point of collection”, filed Aug. 6, 2021, U.S. Provisional Application 63/274,439 entitled “System and method of bioaerosol detection”, filed Nov. 1, 2021, and U.S. Provisional Application 63/328,058 entitled “System and method of nucleic acid synthesis, detection and airborne molecule detection”, filed Apr. 6, 2021. The aforementioned applications are hereby incorporated herein by reference in their entirety.

### TECHNICAL FIELD OF THE INVENTION

The present invention relates generally to system and method of biochemical detection, airborne molecules detection and sample preparation in a resource limited area for point-of-collection.

### BACKGROUND OF THE INVENTION

There has been a growing interest in the point-of-collection (POC) for biochemical assays such as nucleic acid amplification test or allergenic assay for pathogen identification, environmental monitoring, sources of foods and identity of biological subjects, especially for the point-of-collection site at home or other resource limited area.

Since all organisms and viruses have biological material such as nucleic acid, protein or polysaccharide with certain specific sequences could be a signature/genetic feature of a virus or an organism. The current disclosure is to use these biomolecules to identify the source of biomolecules.

As taught in the application (U.S. Ser. No. 17/395,60), the nucleic acid amplification system moves the reaction chambers in the proximity of heat sources for maintaining the reaction temperature, which is according to the stages of amplification reaction or sample preparation. Therefore, by moving the chambers in a system, a nucleic acid amplification reaction can be controlled. The system may be applied to other biochemical reaction or procedure for various antigenic activity assay, nuclei acid amplification assay, nucleic acid library preparation.

Additionally, the system can further adopt more automatic steps to facilitate the biochemical procedures or prepared a nucleic acid library for downstream sequencing. There are only a few of point of collection solutions which allow sample preparation, nucleic acid amplification and/or nucleic acid library construction. Many current solutions require manually pipette and transferring liquid between reaction vessels. It is desired that a system which can perform various biochemical reactions such as purification and/or nucleic acid amplification and/or downstream biochemical analysis with less manual procedures and steps in a POC manner.

Monitoring airborne biomolecule usually requires collection of a bioaerosol sample outside a laboratory but detection of presence of target organism or virus at a laboratory. The whole process usually is time consuming. It is desired that such system can be used to detect source of bioaerosol from environment DNA (eDNA) in a POC manner. Additionally, the nucleic acid in a human/animal's breadth can be used for diagnostic or identification. And the system can prepare a nucleic acid library from the bioaerosol for downstream sequencing.

One way to determine the presence of target nucleic acid in a point-of-collection setting is use of colorimetric method by nucleic acid amplification reaction. However, adding a halochromic agent to a lyophilized reaction mix of nucleic acid amplification increase the complexity of assay optimization process.

Since lyophilized reagents for nucleic acid amplification would address some storage issue in point-of-care setting. It is desired that using a colorimetric method and pH sensitive dye molecules with weak or low buffering capacity of reagents to detect the presence of target nucleic acid as taught in (U.S. Pat. No. 10,253,357B2, Colorimetric detection of nucleic acid amplification, U.S. Pat. No. 9,580,748B2Detection of an amplification reaction product using pH sensitive dyes) but without directly introducing a halochromic agent into reaction mix, which minimizes the complexity of formulating reagents.

### SUMMARY OF THE INVENTION

The present invention advantageously fills the aforementioned deficiencies by System and method of biochemical molecule synthesis and detection in a point of collection setting.

The present invention allows detection of the target organisms or virus with a series of biochemical procedures, which are performed automatically in a POC manner. One examples of the biochemical procedures is nucleic acid library preparation for a target organism.

The present invention also simplifies the formulation of nucleic acid amplification reaction reagents when intended to use pH value of reaction to determine the presence of target nucleic acid. In addition, the present invention provides an avenue to perform detection/synthesis of airborne molecules in a manner of point of collection.

In this regard and for the sake of clarity, a laptop computer might be covered under the definitional use of the term mobile device. A system would not necessarily be covered under the definitional use of the term “portable” or “mobile” by an accompanying apparatus that might give it portability or mobility. Thus, the term “portable” or “mobile” will be used herein (including the claims) to mean devices or system as discussed within this invention.

As used herein, the term “mobile device” means a mobile apparatus that is capable of running a programmed application suitable for executing the embodied functionality. While suitable traditional smart phones may include products such as, e.g., the iPhone, iPad (Apple, Inc.), Android-based devices, Windows, Linux—based device and other well known devices and associated operating systems, the term mobile device as discussed and embodied herein is intended to include any digital mobile device such as smartphones, tablets, phablets, smart watches, smart glass and other current or future smartphone platforms having similar minimal functionality.

It should be understood that the term “adjacent” (and in the claims) does not require that the reaction chamber be in directly contact with the heat source.

The term “suitable” may be used herein (and in the claims) means having the qualities that are correct, needed, or appropriate for something, especially as a person skilled in the art would understand.

As used here “driven”, “translocate” or “shuttle” shall include any form of drive mechanism or facilities for inducing motion in an embodiment. It includes a combination of motor or gears and arms, and the source of driving energy can be one or a combination of electric or mechanic or chemical energy.

As used here, “arm” shall include a linkage that may include one or more arms or leg members, bearings, and one or more receptacles for holding or gripping reaction chambers.

As used herein, the term “air sampler” include but not limit to filters, impingers and impactors, cyclone sampler, liquid impingers, slit samplers and electrostatic precipitators. The collector of an air sampler component is usually where the sample is deposited. As used herein, the term “collector” includes medium. Thus, as used herein (include in the claims), the unlimited examples of the collector of an air sampler component include one or more of followings: water, a buffer, a container, a reagent, a filter, agar or a film.

In current invention, the means for fluid transfer comprise: a variety of fluidic elements such as valves, pistons, plunger, membranes, cantilevers, and the like. The means for fluid transfer cause fluid transferred between two or more different reaction chambers of a system or fluid released from a reagent storage to a reaction chamber. An unlimited example of fluid transfer means comprises one or more of combination: a tube, a channel, valve, a tip, a pump, bulb or a syringe and capillary or melting down a wax bead, which store reagent from a reaction chamber.

In current invention, the term “electronic circuit board” refers to printed circuit board (PCB) or printed wire board (PWB) with integrated circuits (ICs). One of exemplary ICs is a microcontroller or a microprocessor. A microcontroller contains one or more CPUs (processor cores) along with memory and programmable input/output peripherals. Program memory in the form of ferroelectric RAM, NOR flash or OTP ROM is also often included on chip, as well as a small amount of RAM. A programmed electronic circuit board is an electronic circuit board eligible to carry a sequence or set of instructions in a programming language. The sequence or set of instructions is used to control electronic components of the system. The unlimited exemplary electronic components of the system are motors and sensors. With following the sequence or set of instructions, the means for fluid transfer, the means for temperature control and the means for shuttling a reaction chamber of biochemical analysis component are coordinated to perform a task on the electronic component level. An unlimited example of such electronic circuit board is Arduino Uno R3 or Raspberry Pi and anything alike. The examples codes or programs are available on https://docs.arduino.cc.

The term “test platform” refers to a reaction chamber optionally with a reagent or reagent storage component; or a combination of the reagents required for nucleic acid amplification or other biochemical reactions, a cartridge, a receptacle or reaction chambers for holding or storing said reagents or reaction. Practical examples of embodied test platforms or test kit include, but are not limited to, various custom or commercially available test kits or cartridge for nucleic acid amplification or immunoassay. In some embodiments, the term of “test platform” can exchange with “biochemical analysis component”. In some embodiments, the term of “reaction chamber” can exchange with “biochemical analysis component”.

The term “light source” refers to ambient light source or light emitted by LED or light bulb or laser with a range of spectrum from 180 nm to 1064 nm.

In the current invention, wherein the indicia of a sample include one or more of factors: proton concentration, metal ion concentration, antigen concentration, pyrophosphate concentration, dye molecules' concentration, amplified nucleic acid concentration, nucleic acid primer concentration, dye concentration or any reactants associated with the amount of amplified nucleic acid;

The term “sample” or “analyte” refers to anything containing target biomolecules. One examples of biomolecules is amplified nucleic acid and/or nucleic acids obtained from a sample of test. An analyte or sample is one or more of following cells, saliva, blood, tissue, liquid, aerosols.

The term “air filter” refers to a device composed of fibrous, or porous materials which removes particulates such as dust, pollen, mold, aerosols, viruses and bacteria from the air. The non-limited examples of air filter have been listed in (Respiratory protection against bioaerosols: Literature review and research needs, Am J Infect Control. 2004 October; 32 (6): 345-354).

In the current disclosure, the types of biochemical reactions include but not limit to nucleic acid amplification, lysis reaction, transcription reaction, reverse transcription reaction, transposase cleavage reaction, restriction enzyme cleavage reaction, click chemistry reaction (or the biochemical reactions are included and taught in Cold Spring Harbor protocols). The reactions also include nucleic acid amplification, immunoassay and cell-free synthetic reaction (Wearable materials with embedded synthetic biology sensors for biomolecule detection, Nature Biotechnology, 39, p1366-13′74, 2021). The unlimited examples of nucleic acid amplification include any reactions involved with isothermal nucleic acid amplification and polymerase chain reaction (PCR).

In current invention, the non-limited examples of detection means or a functional module include or comprises one or more of following combination: a camera, naked eye, fluorimeter, UV spectrometer, potentiometric sensor, nucleic acid sequencer, fluidic test cassette (as taught in Fluidic test cassette, US 2018/0304260A1), lateral flow device and a pH test strip. The signals used to determine the indicia of nucleic acid amplification reaction include but not limit to fluorescence, UV VIS, voltage and color changes.

In current invention, in one embodiment, the product of nucleic acid amplification is detected by a method or device as a detection module such as a camera, a gel electrophoresis, lateral flow device, fluorimeter, UV-VIS spectrometer, colorimetric method, potentiometric sensor, naked eye and sequencer.

In the current invention, the types of the air samplers include but not limits to single-stage impactors, cascade impactors, cyclones, impingers, electrostatic precipitators and filters.

In the current invention, the means for shuttling biochemical analysis component/reaction chamber is one or more of combinations: arms, linkages, gears, belts or similar facilities and driven by one or more motors. The shuttling operation causes the relative movement of a biochemical analysis component/reaction chamber to other part of a system.

In one embodiment, biochemical analysis component comprises a reaction chamber or a test platform.

The present invention provides a system that detects the presence of target organism or virus from the analyte. The system comprises of an electronic circuit board, at least one reaction chamber, a means for temperature control of biochemical reaction in the reaction chamber, means for fluid transfer, means for shuttling a reaction chamber, a reagent storage component, a functional module and a mobile device.

The reaction chamber of system has an opening to receive an analyte and conducts a biochemical reaction within the reaction chamber. The means for fluid transfer delivers the reagent between a reagent storage component and a reaction chamber or between reaction chambers. The means for temperature control maintains the temperature of biochemical reaction within the reaction chambers. With the means for shuttling a reaction chamber, the reaction chamber is shuttled to a predetermined location of a system for various operations or manipulations. Thereby one of following manipulations/operations is performed for the reagent or analyte inside the chamber: fluid transfer, temperature change, detection of reaction, extraction of nucleic acids, purification. And the functional module is a component or device to carry out one of said manipulations or operations. These manipulations are for completing various stages of biochemical reactions. The means for shuttling a reaction chamber, means for fluid transfer and means for temperature control are controlled by a programmed electronic circuit board. The program is a predesignated process, with the operation of means for shuttling a reaction chamber and means for fluid transfer, to add a reagent from reagent storage component at a predetermined step, to facilitate the biochemical reaction or procedure inside the reaction chamber with means for temperature control or functional module in order to convert the analyte into a biochemical product, which is further detected or sequenced. The programmed electronic circuit board is linked wired or wireless to a mobile device which provides an interface to update the program, record the data as well as transferring the data to internet. An non-limited example of such a stage of biochemical reactions includes annealing of primers, synthesis of nucleic acids, denaturation of nucleic acids, lysis of cells, reverse transcription.

In one embodiment, the means for shutting reaction chambers of the system comprises a motor or actuator. Through a linkage of a motor or actuator and receptacle of reaction chambers, the system shuttles the reaction chamber over different predetermined positions of the system.

In one embodiment, the system shuttles a reaction chamber relatively from contacting a heat source at one predetermined temperature to other heat source at a different predetermined temperature, and the reaction chamber contacts with the heat sources to have a thermal communication, reaching to the predetermined temperature, for a time interval in order to complete a reaction stage or procedure.

In one embodiment, the system comprises means for transfer fluid between the reaction chambers or reagent storage components. Thereby, the product of a reaction in a reaction chamber is transferred to another reaction chamber for a reaction stage or procedure.

In one embodiment, the reagent, buffer or liquid is stored at a reservoir or reagent storage component and is transferred to a biochemical analysis component or reaction chamber by means for fluid transfer.

In one embodiment, the system comprises means for temperature control. The means for temperature control comprises one or more combinations of heat sources, thermoelectric cooling elements and temperature sensor.

In one embodiment, the temperature of reaction chambers is monitored by the thermal chromatic material with its images.

In one embodiment, a temperature sensor is connected to the electronic circuit board and used to determine the reaction chamber's temperature. And the temperature sensor is positioned adjacent to a reaction chamber and have thermal communication with a heat source.

In one embodiment, the program of electronic circuit board is connected to a mobile device wired or wirelessly from internet or locally. And the electronic circuit board is programmed to control one or more operations with followings: means for transfer fluid, means for shuttling reaction chambers/biochemical analysis components, means for temperature control (both for heat sources or thermoelectric cooling elements). The program is updated by the Liquid Crystal Display (LCD) user interface or an interface on a mobile device. The non-limited examples of connecting or linking methods between the mobile device and the electronic circuit board is one of more ways of telecommunications: Bluetooth, wifi, universal serial bus, serial interface (i.e. RS232, RS422,RS485) and ethernet, with their corresponding communication protocols.

In one embodiment, the electronic circuit board has a processor to generate the signals to control the motion of a motor or temperature of a heat source. In one embodiment, there is another electronic circuit board as a motor driver and conveying the signals from a controller circuit to a motor.

In one embodiment, there is an interface on the mobile device so that a user changes or updates the programs or protocols for nucleic acid amplification or sample preparation procedures. In addition, the interface instructs the user to setup the system, design protocols, prepare samples, carry out the nucleic acid amplification reaction and upload/download the system's status information, reaction results, manifest data to cloud.

In one embodiment, the functional module is a magnet. The ligand binding magnetic beads with ligand labeled primers are used to capture the target/amplified nucleic acid in the reaction solution for purification. In one of purification procedure, the system shuttles a reaction chamber to the proximity of the magnet in order to separate the solution and nucleic acid which is hybridized with primers and the primers are bound to the magnetic beads. In one embodiment, the function module is a bead homogenizer or dipstick for nucleic acid extraction (Rapid (30-second), equipment-free purification of nucleic acids using easy-to-make dipsticks, Nature Protocols volume 15, pages3663-3677 (2020)).

In one embodiment, the functional module is an air sampler.

In one embodiment, a lateral flow device is a functional module and used to determine the amplified nucleic acid (Loop-mediated isothermal amplification-lateral-flow dipstick (LAMP-LFD) to detect Mycoplasma ovipneumoniae, World J Microbiol Biotechnol. 2019; 35 (2): 31). The lateral flow device contains a reactive test region and control region.

In one embodiment, a primer conjugated to nucleic acid template is through click chemistry.

The non-limited examples of an operation or procedures include extraction, purification and amplification, detection, library construction such as injection of ligand binding magnetic beads into a reaction chamber, adding proteinase into a reaction chamber to break down the proteins of a sample, adding ligase to connect two nucleic acid fragments, adding click chemistry active primers to the template nuclei acids in a reaction chamber, transferring an reaction intermediate to another reaction chamber, determining the amount of amplified nucleic acid.

One embodied method is further characterized by the following illustrative, exemplary, non-limited aspects, features, or steps: providing (a) at least one reaction chamber to receive said sample; (b) nucleic acid amplification reaction reagents from a reagent storage component. The sample and nucleic acid amplification reaction agents cause nucleic acid amplification reaction to produce amplified nucleic acid; (c) the means for temperature control comprising a heat source/thermoelectric cooling element and a temperature sensor; (d) the means for shuttling the reaction chambers to predetermined positions. The positions are either adjacent to said at least one of heat sources or other positions for operation of a functional module; (e) the means for fluid transfer; (f)at least one programmed electronic circuit board controls the means for shuttling reaction chambers, and the means for temperature control; (g) at least one mobile device connected to the electronic circuit board; introducing the sample and nucleic acid amplification reagent into the reaction chamber, adding reagents and sealing the reaction chamber via means for fluid transfer; controlling the temperature and duration of the reaction chamber for the nucleic acid amplification reaction via the means for shuttling the reaction chamber to the proximity of the heat source and/or changing the temperature of heat source to a predetermined temperature via the means for temperature control, wherein the heat source has thermal communication with the reaction chamber when being adjacent to said heat source, thereby the temperature of the reaction chamber is controlled by shuttling the reaction chamber adjacent to heat source having a predetermined temperature, wherein a duration of the temperature of said reaction chamber is controlled by the means for shuttling reaction chambers via holding said reaction chamber adjacent to the heat source for a predetermined time interval; shuttling said reaction chamber to a predetermined position for collection of said amplified nucleic acid or to a predetermined position for the functional module to carry out its operation; wherein the means for temperature control, the means for shuttling the test platform and the means for fluid transfer are controlled by a programmed electronic circuit board; wherein the programmed electronic circuit board is connected to a mobile device and the program of electronic circuit board is updated by the interface of a mobile device.

In one embodiment, the method further comprises the means for fluid transfer dispense buffers and reagents from the reagent storage component to mix with the analyte. A nucleic acid amplification starts at receiving an analyte. The nucleic acid molecules are released from analyte via heating the reaction chamber or the test platform with a heat source for the lysis stage. The means for fluid transfer further dispense ligand label primers from the reagent storage component to the reaction chamber via means for fluid transfer for the primer hybridization stage. The mix is transferred to second reaction chamber by the means for fluid transfer, the second reaction chamber is further added with magnetic beads by the means for fluid transfer. The second chamber is further transferred to a predetermined location, causing the second reaction chamber adjacent to a magnet. The magnet is a functional module and separates the interested nucleic acids from solution. Once the solution in the second chamber is removed by the means for fluid transfer. A buffer and PCR reaction mix with ligand is added into the second chamber to release the ligand labeled primers and the captured nucleic acids from the magnet. The mix with the releasing ligand labeled primers and the captured nucleic acid is further transferred to the third chamber. The third chamber is shuttled over a different heat source to cause the change of temperature inside the third reaction chamber. The third reaction chamber moves to the first heat source and remains over 95 degree C. for DNA denaturation. And then the third chamber is shuttled to second heat source to cause the temperature remaining at 55 degree C. for annealing. The third reaction chamber further move to third heat source to remain at 72 degree. The cycle of moving third reaction chamber repeats 40 times to finish the nucleic acid amplification.

In one embodiment, the user interface includes an optional time stamping, determining selected quantitative indicia of the sample and storing the determined value for future access; location stamping the determined selected quantitative indicia of the sample and storing the determined value for future access; storing the time and/or location data in at least one of a readable file in the mobile device, an external readable file, and in a cloud file; determining a temporal and/or a location trend of a plurality of the determined selected quantitative indicia of the sample; correlating the determined selected quantitative indicia of the sample to a related selected metric and displaying a value of the related selected metric on the mobile device;

In one embodiment, the nucleic acid amplification reactions include polymerase chain reaction (PCR)/loop-mediate isothermal amplification (LAMP), the result of PCR and LAMP is determined by the color change of reaction product via said mobile device (as taught in U.S. Pat. No. 9,787,815B2, Smartphone-based apparatus and method, U.S. Pat. No. 9,445,749B2, Smartphone-based apparatus and method for obtaining repeatable, quantitative colorimetric measurement.)

In one embodiment, a system for detecting airborne biomolecules, the system comprises at least an air sampler component, a biochemical analysis component, a reagent storage component, and means for fluid transfer, and a heat source. The air sampler component collects bioaerosols from air, and the airborne biomolecules in the bioaerosol are analyzed and/or amplified by the biochemical analysis component. And the reagent storage component stores at least one reagent or buffer for carrying the bioaerosols and for biochemical reaction. The means for fluid transfer dispenses at least one reagent or buffer from the reagent storage component. The at least one reagent or buffer carries the bioaerosols collected on a sampling medium/collector of the air sampler component to the biochemical analysis component, and/or react with bioaerosols in the biochemical analysis component. The heat source maintains the temperature of biochemical reaction in the biochemical analysis component. In one embodiment, the biochemical analysis component further comprises a detection module to determine the reaction at the biochemical analysis component.

A system for detecting airborne biomolecules, the system comprising: (a) one air sampler component which has a collector; (b) an air pump; (c) a biochemical analysis component; (d) biochemical reagents or buffers, which are stored at (e) a biochemical reagent storage component; (f) means for fluid transfer, which are one or more combinations of followings: tubes, channels, pumps or anything alike (g) a heat source; (h) a detection module; wherein the air sampler component is connected to said air pump, thereby the airborne molecules are forced to flow into and be captured by the collector of the air sampler component; wherein the biochemical reagent and/or buffer from the biochemical reagent storage component carries and/or mixes with the airborne molecules via the means for fluid transfer, thereby, the airborne biomolecules react with the biochemical reagent at the biochemical analysis component, and produce a reaction product through one or more reaction stages; one of non-limited examples of such reaction is PCR or LAMP; wherein each of the reaction stage is set on a predetermined temperature; wherein the predetermined temperature is maintained by the heat source; wherein the reaction product is detected by the detection module.

In one embodiment, before the air flow into the collector of air sampler via the inlet, the system comprises a mesh filter to control flowing airborne particles with a predetermined range of sizes.

In one embodiment, the air sampler component of the system comprises an air inlet, air outlet, a sampling medium/collector and an air mover. The air mover is a pump or blower, which directs air flow into the sampling media/collector through the inlet. The bioaerosols/airborne molecules are collected by the media/collector. The air then passes through the system by an outlet.

In one embodiment, a collection chamber of an air sampler is used as a sampling medium/collector, the biochemical analysis is directly performed on the collector of an air sampler component as a reaction chamber or biochemical analysis component.

In one embodiment, an air sampler's filter is a medium/collector, the airborne particles on an absorbance substance of a filter is immersed or rinsed with a buffer or reagent. The rinsing out buffer or reagent containing airborne molecules is further processed by a biochemical analysis component.

In one embodiment, immersed filter with the buffer/reagent is further heated up by a heat source for facilitating lysis or dissolving airborne molecules into the lysis buffer or reagent.

In one embodiment, the sampling medium/collector is an absorption substance such as a filter, or a collector such as a tube for cyclone air sampler (Air-sampling device and method of use, U.S. Pat. No. 7,370,543B2). Once the bioaerosol collection is complete, a reaction chamber has fluid communication with the collection medium/collector, and the fluid carries the air borne molecules into a biochemical reaction chamber.

One of non-limited examples for extraction of nucleic acid from filter is taught in (The Airborne Metagenome in an Indoor Urban Environment, PLOS ONE, Apr. 2, 2008, Efficiency of bioaerosol samplers: a comparison study, Aerobiologia volume 37, pages 447-459 (2021))

In one embodiment, the filter comprises polypropylene or cloth.

In one embodiment, the airborne particles are condensed into at least one liquid drop by a cooler or are captured by a Bubbler (Efficient Detection of Severe Acute Respiratory Syndrome Coronavirus 2 (SARS-CoV-2) from Exhaled Breath, Journal of Molecular Diagnostics VOLUME 23, ISSUE 12, P1661-1670, Dec. 1, 2021). Thereby, the airborne molecules are collected by condensation, and further carried into a biochemical reaction chamber.

In one embodiment, the system further comprises means for shuttling a biochemical analysis component or test platform. The biochemical analysis component or test platform comprises a plurality of chambers situated on a receptacle for reagents and samples, and are composed of a thin glass or plastic or anything suitable for a heat communication with a heat source and collecting bioaerosols. In one embodiment, part of a biochemical analysis component is transparent to facilitate the detection with optical device or naked eyes.

In one embodiment, a plurality of chambers is hold by one or more receptacles, and the receptacles with chambers are the test platform.

In one embodiment, a sampling medium/collector of the air sampler component has a fluid communication with biochemical analysis component and reagent storage. The means for fluid transfer facilitate the fluid communication with channels or tubes. Thereby the airborne biomolecules of bioaerosols are carried into a biochemical analysis component from an air sampler component by a buffer or reagent for the following biochemical reaction.

In one embodiment, the fluid communication between biochemical analysis component and reagent storage is through melting wax beads.

In one embodiment, a reaction chamber receives airborne particles through a fluid communication with sampling medium/collector such as rinsing filters or electrostatic precipitators with at least one reagent or buffer transferred from the reagent storage component. The airborne molecules on an absorbance substance in a filter or electrostatic precipitator are rinsed out by reagents/buffers or dissolve in reagents/buffers, and further being carried into the reaction chamber.

In one embodiment, one or more lysis buffer or reagents are used to extract the nucleic acids of bioaerosols from sampling medium/collector. The non-limited examples for the lysis reagent preparation are taught in (Cold Spring Harbor Protocols).

In one embodiment, a collector of air sampler component is used as a reaction chamber for biochemical reaction such as nucleic acid amplification reaction including or immunoassay, cell-free synthetic reaction (wearable materials with embedded synthetic biology sensors for biomolecule detection, Nature Biotechnology, 39, p1366-1374, 2021).

In one embodiment, the reaction used in biochemical analysis includes polymerase chain reaction (PCR), isothermal nucleic acid amplification reaction (as taught in Isothermal Amplification and Ambient Visualization in a Single Tube for the Detection of SARS-CoV-2 Using Loop-Mediated Amplification and CRISPR Technolog, Anal Chem. 2020 Dec. 15; 92 (24): 16204-16212; Loop-mediated Isothermal Amplification of DNA (LAMP): A New Diagnostic Tool Lights the World of Diagnosis of Animal and Human Pathogens: A Review, Pakistan Journal of Biological Sciences, 17: 151-166.) and reverse transcription (Efficient Detection of Severe Acute Respiratory Syndrome Coronavirus 2 (SARS-CoV-2) from Exhaled Breath, Journal of Molecular Diagnostics VOLUME 23, ISSUE 12, P1661-1670, Dec. 1, 2021).

In one embodiment, the aerosol biomolecules contain a nucleic acid sequence, and the sequence associates with a particular organism of virus.

In one embodiment, the airborne molecules are collected by an impinger. After collection, the liquid in the impinger is dispensed into a reaction chamber for sample preparation or biochemical analysis such as nucleic acid amplification or immunoassay or cell-free synthetic reaction (Wearable materials with embedded synthetic biology sensors for biomolecule detection, Nature Biotechnology, 39, p1366-13′74, 2021).

In one embodiment, the biochemical analysis component of a system has plurality of test platforms. One of the test platforms is configured to receive bioaerosol samples collected for a set time intervals and at least one reagent. Once a collection of an air sample is complete, the test platform is replaced with other test platform by shuttling the other test platform from one position of the system to a predetermined position to receive collected bioaerosol samples, via the means for shuttling biochemical analysis component, for biochemical analysis. While the air sampler component of the system continuously keeps collecting bioaerosols from the environment into the other test platform, the fluid transfer means automatically dispense buffer or reagent from a biochemical storage component and load the collected bioaerosols from the collector into the biochemical analysis component. Thereby, each test platform receives a aerosols sample over a set time interval. The reaction in biochemical analysis component is to determine the source of airborne biomolecules, and the reaction temperature inside the biochemical analysis component is maintained by a heat source. Herein, with a plurality of biochemical analysis components or test platforms, the system repeatedly replaces the biochemical analysis components and determines the presence of target airborne biomolecules in the biochemical analysis component with a detection module or an optional functional module. Thereby, the air sampler components constantly collects aerosol and airborne biomolecules. The captured aerosol biomolecules are automatically transferred, at a set time intervals, to a biochemical analysis component that uses a nucleic acid amplification technique or immunoassay to determine if target airborne biomolecules present in the collected aerosol sample. In one embodiment, the mean for fluid transfer, a mean for shuttling biochemical analysis component and a heat source are controlled by a programmed circuit board. In one embodiment, the electronic circuit board is accessed by internet wirelessly or wired. In one embodiment, the electronic circuit board is connected to a mobile device and the program is updated by the interface of a mobile device.

In one embodiment, a system for detecting airborne biomolecule, the system comprising: one air pump; one air sampler component having a collector for receiving or collecting airborne biomolecules; biochemical reagents or buffers; a plurality of biochemical analysis components at predetermined positions; one biochemical reagent storage component; one heat source; means for fluid transfer carrying the airborne biomolecules or reagents of the system to the biochemical analysis component; means for shuttling the plurality of biochemical analysis components; one detection module or functional module for amplified nucleic acid or immunoassay; a programmed electronic circuit board with a series or set of instructions; wherein the plurality of biochemical analysis components are configured to receive airborne biomolecules collected for a set time interval and the biochemical reagent or buffer; wherein when a collection of airborne biomolecules for a set time interval is complete for one of the plurality of biochemical analysis components, the biochemical analysis component is replaced with other biochemical analysis component by shuttling the other biochemical analysis component from one position of the system to a predetermined position to receive said collected airborne biomolecules, via the means for shuttling biochemical analysis component, thereby, the air sampler component keep collecting airborne biomolecules for the other biochemical analysis component; wherein, the fluid transfer means automatically dispenses the buffer or reagent from the biochemical storage component and load the captured airborne molecules from the air sampler component to the biochemical analysis component, thereby, the collected airborne molecules reacts with the biochemical reagent or buffer from the biochemical analysis component; wherein the reaction temperature inside the biochemical analysis component is maintained by the heat source, thereby the reaction is conducted at a predetermined temperature; wherein the source of airborne biomolecules in the biochemical analysis component is determined by the reaction through the detection module or functional module; thereby, with a plurality of biochemical analysis components, the system constantly replaces the biochemical analysis component and determines the presence of target biomolecules in the biochemical analysis component with said detection module for plurality of time intervals.

A method for detecting airborne biomolecules, the method comprising the steps of: providing (a) an air pump; (b) an air sampler component having one or more collectors;(c) a biochemical reagent or/and buffer;(d) a heat source with a predetermined temperature suitable for a stage of biochemical reaction; (e) a detection module; collecting airborne biomolecules from environment by connecting the air pump to the air sampler component; thereby, the airborne biomolecules are captured by the one or more collector of the air sampler component; introducing the biochemical reagent or/and buffer to the captured biomolecules on the one or more collector of the air sampler component; thereby, the captured biomolecules are mixed with the biochemical reagent; maintaining the predetermined temperature by the heat source; thereby, the biochemical reagent reacts with the captured biomolecules and produces an amplification reaction product for detection; and detecting the amplification reaction product with the detection module.

A method for processing a bioaerosol sample, the method comprising the steps of: providing (a) an air sampler component that receives or collects a bioaerosol sample; (b) a reagent storage component; (c)a test platform or biochemical analysis component that includes at least one chamber to receive at least said one bioaerosol sample and reagents for a nucleic acid amplification reaction or immunoassay, wherein said bioaerosol sample and said reagents cause either nucleic acid amplification reaction or immunoassay to produce amplified nucleic acid or signals, respectively; (d) at least one heat source; (e) at least one detection module or a functional module for amplified nucleic acid or immunoassay; (f) means for fluid transfer carry a sample or reagents of the system to the biochemical analysis component or test platform; introducing the bioaerosol sample into one chamber with a buffer or reagent from the reagent storage component via means for fluid transfer; conducting the amplification reaction at the biochemical analysis component; detecting or having operation on nucleic acid amplification/immunoassay product. In one embodiment, the system further comprises means for shuttling the biochemical analysis component or test platform to predetermined positions of the system; Thereby, detecting or having operation on nucleic acid amplification/immunoassay product at predetermined positions of the system via shuttling the chamber to a suitable position for detection or operations with a detection module or functional module;

In one embodiment, the predetermined positions are suitable for an operation, wherein the operation include one or more of operations: collecting at least one bioaerosol sample, receiving a reagent for biochemical reaction, performing a biochemical reaction with the heat source, detecting the nucleic acid amplification reaction or immunoassay or collection of the nucleic acid amplification reaction product;

In one embodiment, the system further comprises a programmed electronic circuit board to control the means for fluid transfer, and means for shuttling a biochemical analysis component to a predetermined position of the system;

In one embodiment, the system further comprises a mobile device connected to the programmed electronic circuit board.

A method for detecting airborne biomolecule, the method comprising the steps of: providing (a) an air pump, (b) an air sampler, (c) a biochemical reagent in a reagent storage(d) a heat source (e) a detection module. Collecting airborne biomolecules from environment by connecting the air pump to the air sampler. Thereby, the biomolecules are collected in the collector/medium of the air sampler. Introducing the biochemical reagent in a reagent storage to the collected biomolecules. Thereby, the biochemical reagent reacts with the collected airborne biomolecules and produces an amplification reaction product for detection. Detecting the amplification reaction product with a detection module or detection means.

In one embodiment, biochemical analysis component or a test platform is a disposable test kit or cartridge;

In one embodiment, in the airborne detection system, the product of nucleic acid amplification reaction in one biochemical reaction chamber is transferred to another biochemical reaction chamber for nucleic acid library construction. In one embodiment, the produced nucleic acid library construction is further sequenced by a nanopore sequencer.

In one embodiment, the air sampler is a cyclone air sampler. The bioaerosols are trapped on the bottom of a cyclone air sampler or/and a filter. The reagent or buffer is dispensed to the bottom of cyclone air sampler and/or used to rinse out the biomolecules trapped on the filter or the bottom of cyclone air sampler via means for fluid transfer. The reagents further cause a biochemical reaction and produce at least one amplification reaction product for detection or downstream process.

In one embodiment, an air pump connects to an air sampler component. The air sampler takes in air from environment. The bioaerosol molecules from the environment or from the breath of an animal/human are captured by an air sampler component. In one embodiment, the biochemical analysis is used for a diagnosis.

In one embodiment, the reagent storage component is a wax bead. The wax bead contains the reagents for biochemical reaction as taught in (U.S. Pat. No. 5,413,924A, Preparation of wax beads containing a reagent for releasing by heating; (US20110159497A1, Freeze-dried compositions for carrying out PCR and other biochemical reactions).

In one embodiment, the reagent contented wax beads set in the biochemical reaction analysis component. Once heating up the biochemical analysis component, the reagents inside the bead is released and reacts with the target airborne molecules.

The non-limited examples of storage component are reagent reservoir made of one or more of materials: wax, plastic, metal and glass. In one of embodiment, the reagent storage component stores one or more of followings: a buffer, nucleic acid synthesis reagents or nucleic acid extraction reagent. The methods or recipe of reagents and buffers for conducting such biochemical reagent is taught in (Cold Spring Harbor Protocols)

In one embodiment, the result of biochemical reaction is determined by means of detection. The detection mean comprises a detection method or module.

In one embodiment, the detection module is a camera or a mobile device, and an image is taken by the camera or mobile device. The image is used for a colorimetric method as taught in (U.S. Pat. No. 9,787,815B2, Smartphone-based apparatus and method)

As taught in (U.S. Pat. No. 7,888,015B2, qPCR using solid-state sensing), an ion sensitive field effect transistors (ISFET) is used as a sensor to determine the proton produced during nucleic acid amplification, which has been applied to detection of various types of isothermal amplification reactions as taught in (Biosensors and bioelectronics, vol 198, p113802, 2022).

In one embodiment, the shift of pH in a nucleic acid amplification reaction is determined by a ISFET sensor for before amplification reaction starts or after the amplification reaction completes.

Or as taught in (U.S. Pat. No. 8,945,912B2, DNA sequencing and amplification systems using nanoscale field effect sensor arrays), the concentration of nucleotide primers is used as indicia for detection with DNAFET.

In one embodiment, the ISFET/DNAFET probe is immersed into the reaction mix after the reaction in reaction chamber has complete.

In one embodiment, the shift of pH in a nucleic acid amplification reaction is determined by a pH test strip (Visual detection of isothermal nucleic acid amplification using pH-sensitive dyes, BioTechniques 58:59-68), which can be followed by a colorimetric method. In one embodiment, adding a halochromic agent in the reaction mix is used to determine the shift of pH value.

In one embodiment, after completing reaction in a biochemical reaction chamber, the reaction mix is dispensed to a pH test strip or a lateral flow device to determine the presence of target nucleic acid.

In one embodiment, a fluorescence dye molecule is used to determine the amount of amplified nucleic acids.

In one embodiment, the reaction mix for nucleic acid amplification reaction has a low or none buffer capacity or a buffer capacity which is equivalent to 1.5 mM to 19 mM Tris in pH 8.0.

In one embodiment, the reaction mix is prepared as taught in (U.S. Pat. No. 10,253,357B2, Colorimetric detection of nucleic acid amplification, U.S. Pat. No. 9,580,748B2Detection of an amplification reaction product using pH sensitive dyes) but does not have any halochromic agent to indicate the shift of pH before reaction starts and reaction ends. The pH value before the reaction starts is predetermined, once the reaction completes, the reaction mix is loaded to a pH test strip, which may indicate a color change and represent pH shift if there is any. The shift of pH for reaction mix between reaction starts and completes indicates that the nucleic acid amplification and the presence of target nucleic acid in a sample. In one embodiment, lyophilized reagents are used for the nucleic acid amplification reaction. The method for preparation of lyophilized reaction mix is taught in (Lyophilized visually readable loop-mediated isothermal reverse transcriptase nucleic acid amplification test for detection Ebola Zaire RNA)

In one embodiment, biochemical analysis component includes an image acquisition module for colorimetric measurement, a heat source, and an optional light diffuser and/or an optional light-diffusing pathway so as to ensure a uniform and repeatable illumination of at least a desired region of the biochemical analysis component, wherein the light source is one of an internal mobile device flash source, an external LED source, a laser or an ambient light source;

In another embodiment, the detection unit is a portable sequencer that links to a mobile device and performs sequencing. One of such portable sequencer is a nanopore sequencer

In one embodiment, the detection method includes a lateral flow. The lateral flow is used to either detect amplified nucleic acid or antigen in reaction (Loop-mediated isothermal amplification-lateral-flow dipstick (LAMP-LFD) to detect Mycoplasma ovipneumoniae, World J Microbiol Biotechnol. 2019; 35 (2): 31).

In one embodiment, wherein the color calibration region includes a plurality of calibration regions, each of which has a different calibration color or calibration region includes control samples that have predetermined concentration of indicia; obtaining both of the color image of the biochemical reaction and the calibration region using a mobile device including an optional light source; displaying the result of nucleic acid amplification or immunoassay on the mobile device or sending out the information to a cloud service;

In one embodiment, it includes an optional time stamping, determining selected quantitative indicia of the sample and storing the determined value for future access; location stamping the determined selected quantitative indicia of the sample and storing the determined value for future access; storing the time and/or location data in at least one of a readable file in the mobile device, an external readable file, and in a cloud file; determining a temporal and/or a location trend of a plurality of the determined selected quantitative indicia of the sample; correlating the determined selected quantitative indicia of the sample to a related selected metric and displaying a value of the related selected metric on the mobile device;

In one embodiment, the system optionally has an internet connection unit which can sent and report to a remote server wired or wirelessly. Furthermore, the system optionally uploads the analysis result of air sample for each time interval to a remote server, and/or records the results locally in the system.

A statistics method is performed to determine the likelihood of true positive result or true negative result.

In one embodiment, there are three or more than three samples as control samples while there are three or more than three samples as treatment samples.

In one embodiment, a t-test or anova is performed to determine the confidence level of true positive or true negative result for samples.

In order to estimate the precision and accuracy of measurement for biochemical reactions, in one embodiment, the measurements or tests are carried out more than three times. And a statistics method such as t-test is performed over these measurement values to ensure difference of samples between treatment group and control group within a certain confidence level.

In one embodiment, the internet connection system may optionally be integrated with a mobile device. The mobile device performs the analysis of bioaerosol component and/or transmits the analysis result to a server.

The DNA/RNA is extracted by the sample extraction function module of the system from any fluid of a sample. The non-limited examples of the sample extraction functional module include one or more of followings: dipstick and homogenizer, a nucleic acid extraction kit/module and an external device

In one embodiment, the PCR is a convective polymerase chain reaction.

In one embodiment, nucleic acid amplification reagents include but not limited to a combination of DNA polymerase and/or reverse transcriptase, nucleotide, reaction buffers, and/or nucleic acid primers for target nucleic acid fragments, and/or control nucleic acid; and sample preparation reagent includes a combination of cell lysis reagents and/or nucleic acid purification reagents. The non-limited examples of the reagents and biochemical methods is taught in (Cold Spring Harbor Protocols)

Furthermore, the software of system associates an information platform which not only identifies the samples or gene expression levels of samples but also provides further information for downstream treatment or management.

In one embodiment, the results of nucleic acid amplification and geographic location information are sent to cloud and the cloud provides recommendation for a user to take actions based on the result or analysis.

The container or each reaction region associates with a tag. A tag is used to further associate a reaction with a sample or amplification primer sets by the software, which provides convenience for user to operate sample preparation and record registration.

In one embodiment, the software is used to monitor the reaction conditions of nucleic acid. The conditions include temperature, amount of synthesized DNA, signal intensities with temperature stamps or time stamps as well as reaction stages.

In one embodiment, the mobile device provides a user interface and instructs a user to operate the system and display or store the reaction conditions and protocols remotely or locally.

In one embodiment, the mobile device provides a user interface to program the temperature of a heat source and position of reaction chamber.

In one embodiment, the mobile device provides a user interface to control the functional module.

In one embodiment, the mobile device provides a default correction or calibration program to adjust the temperature of a reaction chamber.

In one embodiment, the software communicates with the electronic circuit board of system in order to control one or more of following components: the mean of shuttling reaction chambers or biochemical analysis component, means of fluid transfer, functional module, detection module and a heat source with a wire connection or wirelessly.

In one embodiment, a heating source is an electric thermostat container.

### BRIEF DESCRIPTION OF DRAWING

FIG. 1 illustrates a portable nucleic acid amplification system;

FIG. 2 is a flow chart of an embodiment method in terms of operational steps, procedures at the biochemical reaction stage for nucleic acid library preparation;

FIG. 3A illustrates a nucleic acid amplification system for detection of airborne nucleic acid. In the system, a cyclone air sampler is used to collect airborne biomolecules;

FIG. 3B illustrates a nucleic acid amplification system for detection of airborne nucleic acid. In the system, a cyclone air sampler with a filter is used to collect airborne biomolecules;

FIG. 3C illustrates the means for fluid transfer comprise two tubes in the nucleic acid amplification system for detection of airborne nucleic acid;

FIG. 4A illustrates the assembly of test platform/biochemical analysis components with carousel of a system for detection of airborne biomolecule;

FIG. 4B illustrates a system for detection of airborne biomolecule with a plural number of collectors of air samples while one of air sampler components is collecting airborne biomolecules;

FIG. 4C illustrates a system for detection of airborne biomolecule with a plural number of collectors of air samples while the air sampler component which collected airborne molecules in FIG. 4B is receiving biochemical reagent injection;

FIG. 5 is a flow chart of an embodiment method in term of operational steps or procedures;

FIG. 6 is images taken for the LAMP reaction of the airborne nucleic acids captured by the device illustrated in FIG. 3A.

While the present invention has been described above in terms of specific embodiments, it is to be understood that the invention is not limited to these disclosed embodiments. Many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

### DETAILED DESCRIPTION OF EXEMPLARY EMBODIMENTS

In one of embodiment, the reaction chamber is an Eppendorf tube without a lid for the biochemical reaction. The reaction chamber is held on a receptacle and receives a nucleic acid sample as an analyte. The means for fluid transfer comprises a polytetrafluoroethylene (PTFE), plastic tube and syringe pump. The PTFE tube is also a reagent storage component. The first PTFE tube stores PCR master mix and connects to the first syringe pump. By moving the plunger of first syringe pump, the stored PCR master mix in the first PTFE tube is transferred to the Eppendorf tube. There is a second PTFE tube connected to a second syringe pump. Once the transfer of PCR master mix to the reaction chamber is complete, by driving the plunger of second syringe PTFE tube, the mineral oil is transferred to the Eppendorf tube to seal the reaction. When the transfer of mineral oil is complete, the Eppendorf tube is heated up to 95 degree C. by first heat source to start DNA denature for a predetermined time interval. The receptacle is then driven by a motor and moves over the second heat source and the temperature of the Eppendorf tube reaches to 55 degree C. for a predetermined time interval for DNA annealing. When the DNA annealing stage is complete, the Eppendorf tube moves to over the third heat source via driving the receptacle with the assembly of motors and gears, and the temperature of Eppendorf tube becomes 72 degree C. for DNA primer extension. The cycle is repeated for 40 times and then the nucleic acid amplification reaction is complete. All movement of each single plunger for the syringe pump is driven by motors. All movement of all motors are controlled by a programmed ARDUNO R3 circuit board. The parameters for the temperature of heat source or movement of motors are programmed by an interface of a mobile device through a Bluetooth connection with HC-05 chip of a ARDUNO Rev3 circuit board. The programmed ARDUNO Rev3 electronic circuit board directly controls all motion of motor. One of non-limited motors used herein is 28BYJ-48.

In one embodiment, the system comprises a cyclone air sampler as an air sampler component, and the collector is also a reaction chamber which serves as a biochemical analysis component (U.S. Pat. No. 7,370,543B2, Air-sampling device and method of use). Four polytetrafluoroethylene (PTFE), Tube 1 mm ID×3 mm OD connected to four syringe pumps (5 ml) serve as both a biochemical storage component and means for fluid transfer. The bioaerosols, containing 6×10\hat{ }5 copies of genomic DNA from M13 bacteriophage, is generated and introduced into air by a mesh nebulizer with 300 ul of water. With an air pump, the air with aerosols is directed into the collector via the inlet of cyclone air sampler and aerosols deposit on the collector. Once the collection is complete, the first syringe pushes the 70 ul of buffer/water in the first PET tube into the collector and/or rinse out the target nucleic acid/bioaerosols on the button of collector. The second syringe transfers 30 ul of the reagent for loop mediate isothermal amplification (LAMP) mixes with the buffer rinsed out from the collector and containing target nucleic acid. The method for preparation of the reaction mix and primers is taught in (Method for synthesizing polynucleotides, U.S. Pat. No. 7,374,913). And then the third syringe transfers 100 ul of mineral oil to seal the reaction. The biochemical reaction chamber contacts with water bath of an electronic thermal tumbler or a heat element at 65 degree C. for 1 hour. The temperature of the water bath is setup on the LCD interface of electronic thermal tumbler. After the reaction is complete, the fourth syringe moves the reagent with 10 ul 100× dsGreen dye solution into the reaction. The change in color against control sample indicates the presence of target nucleic acids in bioaerosols.

In one embodiment, the system comprises an air sampler with a polypropylene filter, and the collector is a reaction chamber serving as a biochemical analysis component. Four PTFE tubes connected to four syringe pumps serving as both a biochemical storage component and means for fluid transfer. The air sampler is connected to an air pump. M13 bacteriophage is added into buffer. The air with aerosols generated from a mesh nebulizer passes through the filter and the bioaerosols are collected on the filter. Once the collection is complete, a PTFE tube dispenses buffer into the collector and immerse the filter, and the mineral oil is added to cover the buffer. The buffer and the immersed filter inside the collector is heated up to 90 degree C. for 5 minutes. Thereby, nucleic acid deposited on the polypropylene or cloth filter is released. Once the temperature of buffer cools down to 65 degree C., the LAMP reaction mix with primers are added into the buffer. The reaction starts and continues at 65 degree C. for 1 hour. After the reaction is complete, the fourth syringe injects the reagent with 0.5 ul 10000× dsGreen dye solution into the reaction. The change in color against control indicates the presence of target nucleic acid in bioaerosol and its hue value is determined by a detection module which is a mobile device.

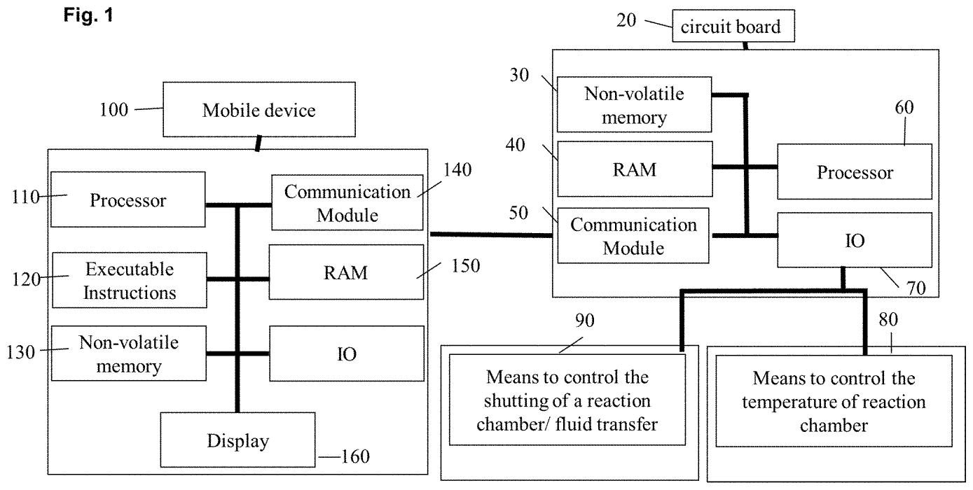

In FIG. 1, the exemplary embodiment shows the system includes a mobile device 100 linked to an electronic circuit board 20. The electronic circuit board 20 connects to the means for shutting of a reaction chambers/fluid transfer 90 and optionally to the means for temperature control of reaction chambers 80. The mobile device may be local or connected to internet. The mobile device may communicate with the means for shutting reaction chambers/fluid transfer 90 or the means for temperature control of reaction chambers 80 through a communication module 50 via serial communications or wireless communication. The means for shuttling a reaction chamber 90 or the means for temperature control of reaction chambers 80 may be under control via a processor 60, and the I.O. 70 may report the status of reaction chambers or temperature of heat sources and/or a reaction chamber via an internal or external bus. A non-volatile memory 30 or RAM 40 may store the programs as execute instructions for translocation or/and temperature control of reaction chamber as well as store all records for the status of system or its components. Further the status of components of system may be assessed from the mobile device 100 as well as the executable instructions may be modified or changed via the display 160 or keyboard of the mobile device. An exemplary embodiment of means for temperature control is a relay which controls the power switch for the heat sources such as heating elements. An exemplary embodiment of means for shuttling of a reaction chamber is an arm driven by a motor.

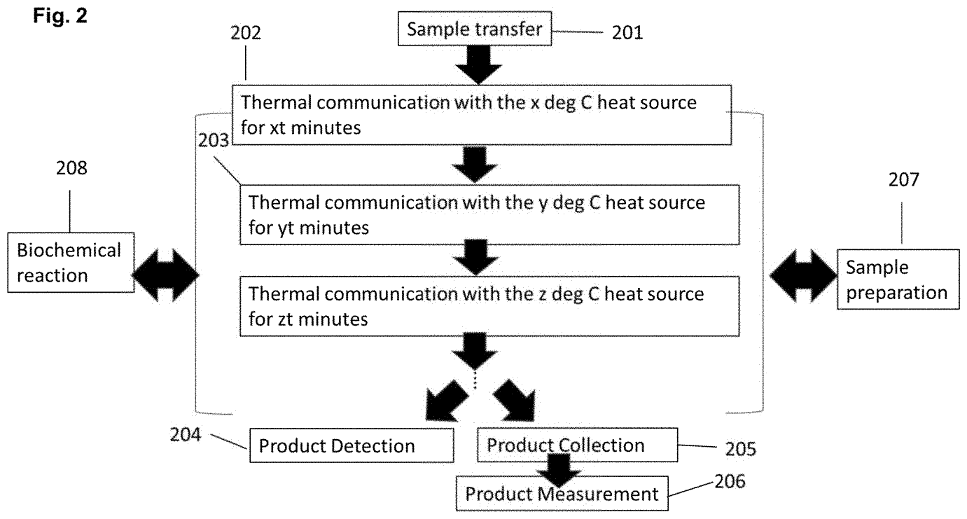

FIG. 2 illustrates a detail workflow of conduct a series of biochemical reactions or procedures in a chamber or test platform. A reaction chamber receives a bioaerosol sample transferred from an air sampler or a liquid sample from a test subject 201, and enters into the biochemical reaction 208 stage. At the biochemical reaction stage 208, the chamber may be shuttled to the proximity of a heat source in order to maintain the temperature of chamber at x degree for xt minutes by means for shuttling the reaction chamber 202. Or instead of shuttling the reaction chamber, the reaction may be transferred into other chamber with x degree for x minute by means for fluid transfer. Once the reaction finishes the time course of xt minutes, the reaction may enter at y degree for other yt minutes via either shuttling the chamber to have thermal communication with other heat source for y degree 203, or transfer the reaction into other chamber with y degree with means for shuttling reaction chambers or means for fluid transfer, respectively. The process may continue with various temperatures and time intervals in order to complete the sample preparation 207 process and/or biochemical reaction 208. Once the biochemical reaction or sample preparation is complete, the reaction product is either detected by a detection module 204 or collected 205 for further measurement 206 such as sequencing.

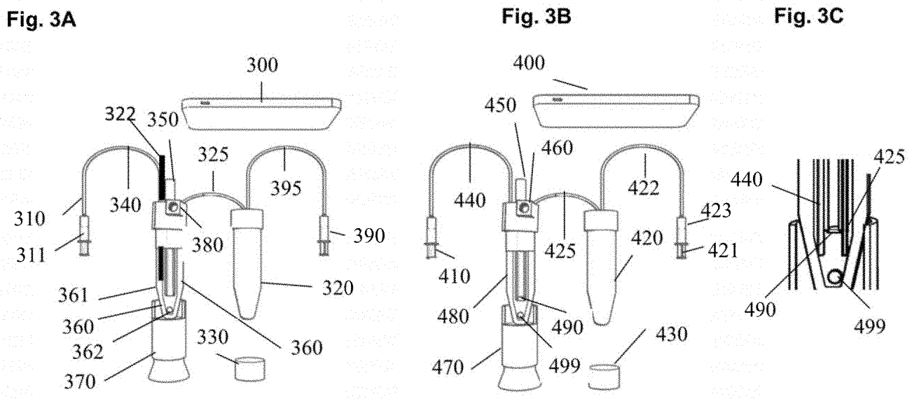

In FIG. 3A, the exemplary embodiment shows the system includes cyclone air sampler 361, a tube 350 connected to an air pump. The air and airborne biomolecules are forced to flow into the cyclone air sampler 361 from the inlet 380 by connecting the tube 350 to an air pump. The flowing airborne biomolecules are captured/collected by the collector of air sampler 360. Once the collection is complete. The reagents from a reagent storage 340 which is a tube, is injected to the collector of air sampler 360 by pushing the plunger of syringe 311. A heating element 370 under the collector of air sampler 360 maintains the reaction temperature. The collector of air sampler 360 is transparent, with introducing dyes, the presence of airborne is determined by the color change against control sample. In one embodiment, the reaction mix in the collector of air sampler 360 can be further transfer to into a reaction chamber 320. Thus, the collector of cyclone air sampler 360 or the reaction chamber 320 serve as a biochemical analysis component while the tube 340 with syringe 311 are means for fluid transfer.

In one embodiment, the biochemical reagent is stored in a wax bead 362, the wax bead is kept on the biochemical analysis component or reaction chamber (a collector of a cyclone air sampler). Upon heating the wax bead 362 with a heating element 370 (a heat source), the biochemical reagent is released to the collector of cyclone air sampler 360 and causes the reaction at 360. Thus, the means for fluid transfer include wax bead 362 and heating in this embodiment. In one embodiment, released reagents from the wax bead 362 with captured airborne biomolecules are further delivered to the reaction chamber 320. Or in another embodiment, the biochemical reagent with captured airborne biomolecules at 360 is transferred via the tube 325 into reaction chamber 320 by connecting another tube 395 to the reaction chamber 320 and pulling the syringe 390. Herein, in the embodiment, the fluid transfer means include tube 325,395 and syringe 390; and biochemical analysis component is reaction chamber 320. In one embodiment, the reagent is in lyophilized form deposited at the collector of cyclone air sampler 360 and a buffer is delivered into the collector of cyclone air sampler 360 to hydrate the biochemical reagents and carry out the biochemical reaction with the captured airborne biomolecules. Once the biochemical reaction is complete, the camera/detector 300 above the cyclone air sampler may take images of reaction with an optional LED light source 330. Therefore, the LED 330 and camera/detector 300 are a detection module. In one embodiment, the color change of biochemical reaction can be observed by naked eyes. Thus, naked eyes are a detection module. In one embodiment, a FET sensor 322 is placed into the reaction mix in the reaction chamber 320 or the collector of cyclone air sampler 360 to determine the final pH of reaction mix while the pH value of reagents at 310 is predetermined. From the shift of pH value, one may determine if the presence of airborne molecules. Thus, FET sensor 322 is a detection module. In one embodiment, a pH test strip dips into the reaction, and the pH value of reaction is determined by the color change. In this embodiment, a pH test strip is a detection module.

In FIG. 3B, the exemplary embodiment shows the system includes a biochemical analysis component 480, which is also a collector of cyclone air sampler and connects to an air pump through a tube 450. There is air filter covers one end of the tube 490. The air containing the airborne molecules is forced to flow into the collector of a cyclone air sampler 480 from inlet 460 by connecting to an air pump. The airborne biomolecules are captured by the filter 490. Once the collection is finished, a biochemical reagent is injected into the biochemical analysis component 480 from tube 440 via pushing syringe plunger 410. The reagent or buffer covers, immerses or rinses the filter 490 and carries out the captured molecules into the reagent. The reagent may cause biochemical reaction for detection. The reaction product is detected in the biochemical analysis component 480 or being delivered to the reaction chamber 420 through the tube 425 by pulling syringe plunger 421, the syringe 423 connects to the reaction chamber 420 via tube 422. Both tube 425, 422 and reaction chamber 420 are sealed. Thus, by pulling the syringe plunger 421, air or fluid can be drew in the reaction chamber 420. Under the chamber 480, there may be an optional heat source 470 which maintain a suitable temperature for the biochemical reaction. The 499 is a wax bead which contains reagents for the amplification reaction. The reaction chamber 420 is transparent, in one embodiment, the reaction chamber 420 is used for detection. The LED light source 430 under reaction chamber 420 is used as a light source for image/signal acquisition by a mobile device or detector 400. In one embodiment, a FET sensor is placed in chamber 420 to determine the final pH of reaction mix while the pH value of reagents at 440 is predetermined.

In FIG. 3C, the exemplary embodiment shows the means for fluid transfer in the system illustrated in FIG. 3B. Within the collector of cyclone air sampler 480, the opening of two tubes 440 and 425 are under one end of tube 450 or the filter 490 so that the dispensed buffer or reagent may cover the filter 490 and can be transferred to reaction chamber 420 via tube 425.

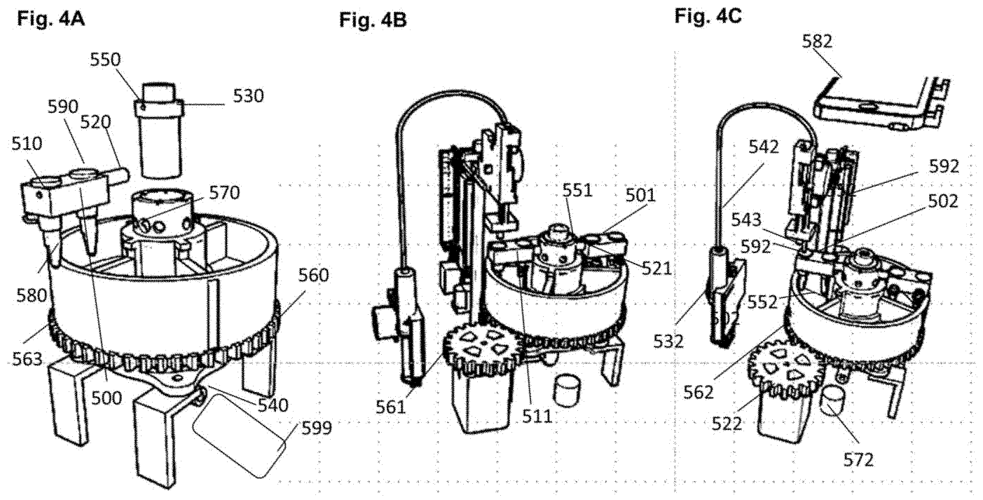

In FIG. 4A, the exemplary embodiment shows the system has a biochemical analysis component (test platform) 500, and the biochemical analysis component contains a wax bead with reagents inside. The biochemical analysis component is also a collector of a cyclone air sampler and has an air inlet 510 and outlet for air flow 520. The center pole of carousel is a tube 530. One end of the tube 540 is connected to an air pump 599. The tube also has one opening 550. When a motor triggers gears 560 to rotate the carousel, the receptacle 570 holding biochemical analysis components is able to rotate and adjust the location/direction of a biochemical analysis component's air outlet toward the opening on the center pole of carousel, which is the tube 530. Once the opening on the center pole 550 and the outlet of biochemical analysis component 520 are aligned, the air from environment flows through the air inlet of collector 510 to the air outlet 520 of collector (when mounted to the center pole of carousel), and then flows into the center pole of carousel via its opening on 550. Eventually, the air leaves the center pole of carousel from the opening 540, which connects to an air pump 599. The airborne particles are captured on the collector 580, which is also a biochemical analysis component or reaction chamber. The collector 580 is sealed with a transparent plastic film 590. A liquid dispenser can pierce the film and inject a reagent or buffer into the collector 580. Also, the film 590 allows a light to pass through and facilitate image acquisition or optical detection as well as the bottom surface of the carousel 563 so that light can pass through the bottom surface of the carousel 563.

In FIG. 4B, the exemplary embodiment shows the system has two biochemical analysis components (test platforms) 501 and 511. The air outlet 521 of first biochemical analysis components 501 connects and aligns with the opening of center pole of carousel 551. The air from environment is forced to flow into the first biochemical analysis components 501 for air sample collection. Once a collection of an air sample is complete, the first biochemical analysis components 501 is replaced with second biochemical analysis component 511 by shuttling the second biochemical analysis component from one position of the system to a predetermined position, via the means for shuttling biochemical analysis component 561, a combination of gears and motors. At this predetermined position, the air outlet of biochemical analysis component 511 and the opening of center pole of carousel are aligned and connected. Thereby, the air flows into biochemical analysis component 511, and the second biochemical analysis component 511 continually collects air sample from environment.

In FIG. 4C, While the air sampler component continuously keeps collecting bioaerosols from the environment into the second biochemical analysis components 511 (which is also a collector of the air sampler component), a liquid dispenser 543 pierces the transparent film 592, and the means for fluid transfer 532—a combination of syringe pump and tube, gears, motors and dispenses the biochemical reagent or buffer from a tube 542, the biochemical storage component, into the first biochemical analysis component 502 at a predetermined location of system. At this predetermined location, there is an oil bath 552 beneath the biochemical analysis component. The first biochemical analysis components 502 is partially immersed at the oil bath 552. Under the oil bath, there is a heating element 562, which maintains the reaction temperature of first biochemical analysis component 502 via heating the oil bath. Once the reaction is complete, the first biochemical analysis component is shuttled to a predetermined position for detecting the amplification reaction result with a LED light source 572 beneath the first biochemical analysis component 502 and a mobile device 582 above first biochemical analysis component. The mobile device takes images of reaction inside first biochemical analysis component 502 and determines the presence of source of biomolecules at aerosols. Thereby, with a plurality of biochemical analysis components, the system has each individual biochemical analysis component to receive a aerosols sample over a set time interval at different time points. Thereby, with a plurality of biochemical analysis components, the system constantly collects the air samples from environment, replaces the biochemical analysis component and determines the presence of target biomolecules in the biochemical analysis component with a detection module or an optional functional module. Wherein, said means for fluid transfer 532, said means for shuttling biochemical analysis component 522 and said heat source 562 are controlled by said programmed electronic circuit board 592. Thereby, the system keeps monitoring bioaerosols in environment. In one embodiment, the programmed electronic circuit board is connected to a mobile device 582 which connects to Internet and provides a user interface for the information of monitoring. In one embodiment, a plurality of biochemical analysis components/collectors of cyclone air sampler collect air sample at the same time for a particular time interval.

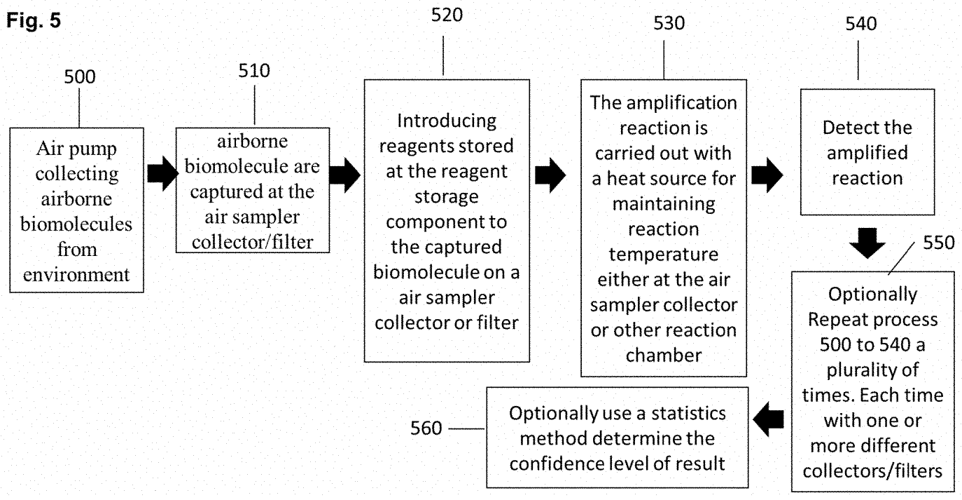

In FIG. 5, the exemplary embodiment methods in the flow chart: providing a system with (i) an air pump, (ii) an air sampler, (iii) biochemical reagent in a reagent storage, (iv) a heat source (vi) a detection module. Collecting airborne biomolecules from environment by connecting an air pump to the air sampler 500. Thus, biomolecules are captured in the collector of said air sampler 510. Introducing the biochemical reagent or buffer in a reagent storage component to the captured biomolecules 520. Therefore, the biochemical reagent reacts with the capture biomolecules at a temperature maintained by the heat source and produces an amplification reaction product for detection 530. Detecting said amplification reaction product with a detection module or detection mean 540. In one embodiment, the detection module is naked eye. In one embodiment, the process repeats a plurality of times 550. Each time with one or more different collectors/filters for air sampling, and the results are analyzed with a statistics method for estimating the confidence levels of results 560. Therefore, the system keeps monitoring bioaerosols in environment.

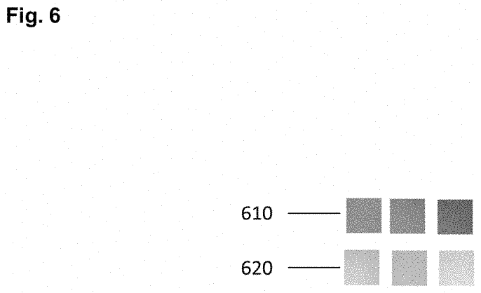

In FIG. 6, the exemplary images are taken from the LAMP reactions with collected airborne biomolecules—bacteriophage M13 genome DNA at two different copies numbers. The images on first row are three bioaerosol samples with no bacteriophage M13 genome DNA 610, and the images on second row are three bioaerosol samples with 6×10\hat{ }7 copies of bacteriophage M13 genome DNA 620. The bacteriophage M13 genome DNA is collected with devices shown in FIG. 3. A blue LED light is used as a light source and the images are taken with a mobile device.

### EXEMPLIFICATIONS

Example 1: In this example, as configured in FIG. 1, the system comprises a mobile device, an electronic circuit board, at least one heat source and one motor driven arm. The electronic circuit board controls the temperature of heat sources and the motors. The electronic circuit board turns on or off the relay for the electricity of heat sources or motors as well. The program of electronic circuit board is updated or changed through a link/connection between the mobile device and electronic circuit board. The user may edit the program on user interface displayed on the mobile device and upload the program to the electronic circuit board. The electronic circuit board controls the arm and heat sources accordingly while the temperature sensors report the temperature to the user on the interface of mobile device via the connection between the electronic circuit board and sensors. In addition, the user interface process and analyzes data (one example of data are the images of reactions taken from mobile device) as well as provides the instruction to guide the user to perform the test or construction of nucleic acid library. The data collected to mobile device is further used for calibration and correction.

Example 2: In this example, as described in FIG. 2, a user can provide a sample to the system or the system automatically collects the sample. A programmed electronic circuit board controls a combination of motors/gears/arms to shuttle Eppendorf tubes (The Eppendorf tubes are reaction chambers/biochemical analysis components.), and push/pull syringe plungers which dispenses/draws a reagent to/from Eppendorf tubes as means for fluid transfer. The electronic circuit board further controls on/off of PCT heating elements. They are the heat sources maintaining predetermined temperatures for biochemical reaction inside the Eppendorf tubes. A series of biochemical reactions or procedures are carried out with a sample. For instance, by holding a sample with an Eppendorf tube over a heat source at x degree C. for xt second, it controls the reaction temperature and duration. It can also shuttle an Eppendorf tube which has a mix of nucleic acids hybridized with primer labeled magnetic beads. When the magnetic beads of Eppendorf tube is attracted by a magnet, the solution the Eppendorf tube can be replaced via a syringe pump and tube (the syringe pump and tube are the means for fluid transfer.) for purification of nuclei acids. The non-limited examples of procedures or reactions are extraction, purification, reverse transcription, amplification, ligation and amplification for a nucleic acid detection or library construction.

Example 3: In this example, as configured in FIG. 3A., the airborne biomolecule detection device collects and captures the biomolecules at the collector of cyclone air sampler. The airborne biomolecules are the target nucleic acid from a virus or organism, a biochemical reagent could be one or more of followings: a buffer, water, primer set, nucleotides, DNA polymerase and nucleic acid amplification reagents, which carries target nucleic acid in bioaerosols to a reaction chamber/biochemical analysis component for nucleic acid amplification or, in one embodiment, the nucleic acid amplification is performed at the collector of cyclone air sampler. In one embodiment, the nucleic acid amplification reaction is loop-mediate nucleic acid amplification reaction (LAMP), and the target nucleic acid is bacteriophage M13. With a device shown in FIG. 3A, bioaerosols are generated by a mesh nebulizer with 3×10\hat{ }4 bacteriophage M13 particles in 300 ul TE buffer solution, and the bioaerosols are directed into 1 gallon enclosed plastic box. The plastic box has two openings. One is for receiving bioaerosols and the other is air inlet of the device shown in FIG. 3A setting. The air pump operates with an air flow rate 625 L/min connected to the cyclone air sampler or the device shown in FIG. 3A. The collection process takes 3 minutes till all bacteriophage M13 solution being aerosolized. The TE buffer is injected into the collector of cyclone air sampler, and carries the bacteriophage M13 nucleic acid.

The reagent prepared according to (Loop-mediated isothermal amplification of DNA, Nucleic Acids Res. 2000 Jun. 15; 28 (12): e63.) is injected into the collector of cyclone air sampler and used to determine the presence of bacteriophage M13 nucleic acid. The amplification reaction is conducted for 1 hour at 65 deg C. After completing the reaction, a 1 ul of 100× dsGreen is added to the reaction mix. A mobile device is used to acquire the images of reactions. The tests were carried out three times. From the change in hue values of reaction images, it shows the presence of bacteriophage M13 in the bioaerosols.

Example 4: In this example, as configured in FIG. 3B, the airborne biomolecule detection device collects and captures the biomolecules at the filter of air sampler. A reagent and/or buffer is injected into the collector of air sampler and rinse out the target nucleic acid from the air filter. The rinsing out target nucleic acid is carried to a reaction chamber via a tube and syringe pump or at the collector of air sampler for nuclei acid amplification as shown in FIG. 3B.

Example 5: In this example, as configured in FIG. 3B, the reagents is stored at a wax bead. Upon heating at the collector of air sampler component, the reagent is released from the wax bead and reacts with the target nucleic acids.

While the present invention has been described above in terms of specific embodiments, it is to be understood that the invention is not limited to these disclosed embodiments. Many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

What is claimed is:

1. A system for processing a sample, the system comprising:
(a) a programmed electronic circuit board;
(b) a plurality of reaction chambers;
(c) means for temperature control;
(d) means for fluid transfer;
(e) means for shuttling the plurality of reaction chambers;
(f) reagents for nucleic acid amplification reaction;
(g) a reagent storage component;
(h) a functional module; and
(i) a mobile device,
wherein said reaction chamber has an opening to receive an analyte and conduct a biochemical reaction within;
wherein said means for fluid transfer delivers the reagent between said reagent storage component and said plurality of reaction chambers or between said plurality of reaction chambers;
wherein said means for temperature control comprising a temperature sensor and a heat source for maintaining the temperature of said biochemical reaction within said plurality of reaction chambers;
wherein said means for shuttling the plurality of reaction chambers shuttles the plurality of reaction chambers to a predetermined location of the system for various operations or manipulations; thereby one of the following operations or manipulations is performed for the biochemical reaction inside said plurality of chambers: fluid transfer, temperature change, detection of reaction, extraction of nucleic acids, purification;
wherein said functional module is a component or device for carrying out one of said operations or manipulations; thereby, said operations or manipulations are for completing various stages of biochemical reactions;
wherein said means for shuttling the plurality of reaction chambers, said means for fluid transfer and said means for temperature control are controlled by said programmed electronic circuit board; thereby, said programmed electronic circuit board performs a series of operations involving said means for shuttling the plurality of reaction chambers, said means for fluid transfer and said means for temperature control to facilitate said biochemical reaction or procedure inside said plurality of reaction chambers in order to convert the analyte into a biochemical product for further detection or sequencing;
wherein said programmed electronic circuit board is linked via wired or wireless connections to the mobile device which provides an interface to update the program, processing reaction results, recording manifest data, and transferring the protocols, reaction results, manifest data to/from internet.

2. The system of claim 1, wherein said functional module is a nucleic acid extraction module including one or more—of the following components: a reaction chamber for cell lysis, a bead homogenizer, a nucleic acid purification module, a column, magnetic bead and dipstick.

3. A system for detecting airborne biomolecules, the system comprising:
(a) at least one air sampler component having a collector;
(b) at least one air pump;
(c) at least one biochemical analysis component;
(d) at least one biochemical reagent or buffer;
(e) at least one biochemical reagent storage component;
(f) means for fluid transfer;
(g) at least one heat source;
(h) a detection module;
wherein said air sampler component is connected to said air pump, thereby said airborne molecules are forced to flow into and be captured by the collector of said air sampler component;
wherein said biochemical reagent and/or buffer from said biochemical reagent storage component carries and/or mixes with said airborne molecules via said means for fluid transfer, thereby, said airborne biomolecules react with said biochemical reagent at said biochemical analysis component, and produce a reaction product through one or more reaction stages;
wherein each said reaction stage is set on a predetermined temperature;
wherein said predetermined temperature is maintained by said heat source;
wherein said reaction product is detected by said detection module.

4. The system of claim 3, wherein said airborne molecules are nucleic acids and said biochemical reagent causes nucleic acid amplification.

5. The system of claim 3, wherein said means for fluid transfer includes one or more of:
a channel, a tube, a capillary, a pump, a syringe, a wax container and heating.

6. The system of claim 3, wherein said reagent storage component includes a wax bead or wax container within said biochemical analysis component, thereby, by heating said biochemical analysis component, said biochemical reagent or buffer is released and mixes with said airborne biomolecules.

7. The system of claim 3, wherein biochemical reagent includes dye molecules, wherein the dye molecules serve as indicia for the amount of amplified nucleic acids.

8. The system of claim 3, wherein the amount of amplified nucleic acids are determined by a FET sensor or a pH test strip.

9. The system of claim 3, wherein said detection module determines the amount of reaction product by at least one of following signals: fluorescence, UV, colorimetric, electrical potential.

10. The system of claim 3, wherein said system further comprise a mesh filter to allow airborne particles with a range of sizes to enter the reaction chamber.

11. The system of claim 3, wherein said air sampler component of system further comprises an air filter as a collector to collect aerosols.

12. The system of claim 3, wherein said air sampler component is a cyclone air sampler.

13. The system of claim 12, wherein said collector of said cyclone air sampler serves as a biochemical analysis component as well, thereby the amplification reaction is within said collector.

14. The system of claim 3, wherein said biochemical analysis component is a fluidic test cassette.

15. A system for detecting airborne biomolecule, the system comprising:
(a) at least one air pump;
(b) at least one air sampler component having a collector for receiving or collecting airborne biomolecules;
(c) at least one biochemical reagent or buffer;
(d) a plurality of biochemical analysis components at predetermined positions;
(e) at least one biochemical reagent storage component;
(f) at least one heat source;
(g) means for fluid transfer carrying said airborne biomolecules or reagents of said system to said biochemical analysis component;
(h) means for shuttling said plurality of biochemical analysis components;
(i) at least one detection module or functional module for amplified nucleic acid or immunoassay;
(j) a programmed electronic circuit board with a series or set of instructions;
wherein said plurality of biochemical analysis components are configured to receive airborne biomolecules collected for a set time interval and said biochemical reagent or buffer.
wherein when a collection of airborne biomolecules for a set time interval is complete for one of the plurality of biochemical analysis components, said biochemical analysis component is replaced with other biochemical analysis component by shuttling said other biochemical analysis component from one position of the system to a predetermined position to receive said collected airborne biomolecules, via said means for shuttling biochemical analysis component, thereby, said air sampler component keeps collecting airborne biomolecules for said other biochemical analysis component.
wherein, the fluid transfer means automatically dispenses said buffer or reagent from said biochemical storage component and load said captured airborne molecules from said air sampler component to said biochemical analysis component, thereby, said collected airborne molecules reacts with said biochemical reagent from said biochemical analysis component;
wherein the reaction temperature inside said biochemical analysis component is maintained by said heat source, thereby said reaction is conducted at a predetermined temperature;
wherein the source of said airborne biomolecules in said biochemical analysis component is determined by said reaction through said detection module or functional module; thereby, with a plurality of biochemical analysis components, said system constantly replaces said biochemical analysis component and determines the presence of target biomolecules in the biochemical analysis component with said detection module for plurality of time intervals.

16. The system of claim 15, wherein said programmed electronic circuit board is accessible by internet via wireless or wired connections.

17. The system of claim 15, wherein said programmed electronic circuit board is linked to a mobile device and said series or set of instructions are updated by the interface on said mobile device.

18. The system of claim 15, wherein said detection module includes one or more of followings: a mobile device, camera, fluorimeter, UV spectrometer, potentiometric sensor, nucleic acid sequencer, pH value stick and lateral flow device, thereby, the reaction results are determined by said detection module.

19. A method for detecting airborne biomolecules, the method comprising the steps of: providing
(a) an air pump;
(b) an air sampler component having one or more collectors;
(c) a biochemical reagent or/and buffer;
(d) a heat source with a predetermined temperature suitable for a stage of biochemical reaction;
(e) a detection module;
collecting airborne biomolecules from environment by connecting said air pump to said air sampler component; thereby, said airborne biomolecules are captured by said one or more collector of said air sampler component;
introducing said biochemical reagent or/and buffer to said captured biomolecules on said one or more collector of said air sampler component; thereby, said captured biomolecules are mixed with said biochemical reagent;
maintaining said predetermined temperature by said heat source; thereby, said biochemical reagent reacts with said captured biomolecules and produces an amplification reaction product for detection; and detecting said amplification reaction product with said detection module.

20. The method of claim 19, wherein detection results from the detection module are analyzed by a statistics method such as t-test for a confidential level of the presence of the airborne molecules.

21. The method of claim 19, wherein said air sampler component collects said airborne biomolecules for one or more of the plurality of collectors, for a set time interval at a time and said collected airborne biomolecules react with said biochemical reagent or/and buffer; thereby, the results of said reaction can determine the presence of said airborne biomolecules.

22. The method of claim 19, wherein said amplification reaction is a polymerase chain reaction or nucleic isothermal amplification reaction.
